(19) United States
(12) Patent Application Publication
(10) Pub. No.: US 2017/0258827 A1
(43) Pub. Date: Sep. 14, 2017

(54) METHOD FOR TREATING CANCER

(71) Applicant: Frederick J. Sawaya, Farmington Hills, MI (US)

(72) Inventor: Frederick J. Sawaya, Farmington Hills, MI (US)

(21) Appl. No.: 15/610,482

(22) Filed: May 31, 2017

(51) Int. Cl. A61K 33/04; A61K 9/00

(52) U.S. Cl. CPC A61K 33/04; A61K 9/0019; A61K 9/0053; A61K 9/0014

(57) ABSTRACT

A method for treating a cancer by administering a therapeutically active composition for the treatment in a patient or human thereof, is disclosed. The method according to the present invention, comprises a step of administering to the human a pharmaceutical composition comprising a therapeutic amount of sodium thiosulfate (Na₂S₂O₃) along with at least one of a pharmaceutical carrier and a pharmaceutical excipient. The carriers, or excipients are selected corresponding to the mode of administration to the patient with cancer includes gastric cancer, fibrosarcoma, pancreatic cancer, and head and neck carcinomas. The method further comprises a step of administering the said composition is any one of oral route, intravenous route, inhalation route, intra-vesical route, vaginal route, rectal route, sublingual route, ophthalmic route and topical route.

### CROSS-REFERENCE TO RELATED PATENT APPLICATION

This patent application is entitled to the benefit of the filing date of provisional U.S. patent application Ser. No. 62/122,273 filed Oct. 17, 2014, under 35 USC 120. This patent application is also a divisional of U.S. patent application Ser. No. 14/707,399, filed May 8, 2015.

### BACKGROUND OF THE INVENTION

A. Technical Field

The present invention generally relates to a method for treating cancer in a patient or human subject thereof. More particularly, the present invention relates to the method for treating cancer by administering to the patient or human a pharmaceutical composition comprising therapeutic amount of sodium thiosulfate (Na₂S₂O₃).

B. Description of Related Art

A virus is a small infectious agent that replicates only inside the living cells of other organisms. Viruses can infect all types of life forms, from animals and plants to microorganisms, including bacteria and archaea. Other viruses such as herpes, dengue, pox, measles, mumps rubella, HIV, zika viruses are made up of glycoprotein membrane, sulfur disulfide bonds and metal atoms. There is no other human safe drug which causes impairment of the glycoprotein components of the membranes of these viruses. Sulfur containing compounds have been proven for their effects on the impairment of S—S bonds and cause inhibition of the virus growth, however, none of the prior sulfur containing compounds are human safe or truly antiviral.

The drug Bactrim is primarily antibacterial for the treatment of routine urinary tract infections. Bactrim contains a combination of sulfamethoxazole and trimethoprim. The Sulfamethoxazole and trimethoprim are both antibiotics that treat different types of infection caused by bacteria only.

Thus there is a long felt but unresolved need for method to treat a cancer by administering a composition which has a quality of impairing the disulfide bonds in viruses and causing the viruses inhibition, mutation, impairment and death of cancer causing virus. This occurs from the cellular level on upwards.

The above mentioned shortcomings, disadvantages and problems are addressed herein, as detailed below.

### SUMMARY OF THE INVENTION

The various embodiments herein provide a method for treating a cancer by administering a therapeutically active composition for the treatment in a patient or human thereof. In an embodiment, the method comprises a step of administering pharmaceutical composition comprising a therapeutic amount of sodium thiosulfate (Na₂S₂O₃) along with at least one of a pharmaceutical carrier and a pharmaceutical excipient. The carriers, or excipients are selected corresponding to the mode of administration to the patient with cancer includes gastric cancer, fibrosarcoma, pancreatic cancer, and head and neck carcinomas.

In an embodiment, the method comprises a step of administering the said composition is any one of oral route, intravenous route, inhalation route, intra-vesical route, vaginal route, rectal route, sublingual route, ophthalmic route and topical route. In one embodiment, the method comprises a step of administering the said composition via intravenous route, is done by infusion of a solution comprising IV-injectable sodium thiosulfate (Na₂S₂O₃). In one embodiment, the method comprises a step of administering the said composition via oral route is in one of a dosage form of tablet, powder, or capsule. In another embodiment, the method comprises a step of administering the said composition via topical route is in one of a dosage form of solution, cream, paste, or lotion.

In one embodiment, the method comprises a step of administering the said composition comprises one or more pharmaceutical acceptable carriers and excipient, wherein the pharmaceutically acceptable carriers and excipients is water. In one embodiment, the method comprises a step of administering of the said composition comprising sodium thiosulfate (Na₂S₂O₃) compound is effective against cancer viruses containing disulfide bonds, wherein the cancer virus containing disulfide bonds are papilloma virus and Epstein-Barr virus (EBV).

In one embodiment, the method comprises a step of administering the said composition comprising sodium thiosulfate (Na₂S₂O₃) compound binds with a polymerase magnesium of a viral metal and viral heavy metal, thereby disabling or killing the virus. In another embodiment, the method of administering the said composition comprising sodium thiosulfate (Na₂S₂O₃) compound releases heavy metal binding atoms and molecules. Further, the method of administering the said composition comprising sodium thiosulfate (Na₂S₂O₃) compound attacks the mucin component of the virus. In another embodiment, the method of administering the said composition comprising sodium thiosulfate (Na₂S₂O₃) provides exogenous electrons for confounding a viral disulfide bond repair. In some embodiments, the administering frequency is daily dosage in the range of 1 mg-2 g per kg body weight of the patient.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DETAILED DESCRIPTION OF EMBODIMENTS

It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

The present invention relates to a method for treating cancer to a patient in need thereof. In an embodiment, the method comprises a step of administering pharmaceutical composition comprising a therapeutic amount of sodium thiosulfate (Na₂S₂O₃) along with at least one of a pharmaceutical carrier and a pharmaceutical excipient. The carriers, or excipients are selected corresponding to the mode of administration to the patient with cancer includes gastric cancer, fibrosarcoma, pancreatic cancer, and head and neck carcinomas.

In an embodiment, the method comprises a step of administering the said composition is any one of oral route, intravenous route, inhalation route, intra-vesical route, vaginal route, rectal route, sublingual route, ophthalmic route and topical route. In one embodiment, the method comprises a step of administering the said composition via intravenous route, is done by infusion of a solution comprising IV-injectable sodium thiosulfate (Na₂S₂O₃). In one embodiment, the method comprises a step of administering the said composition via oral route is in one of a dosage form of tablet, powder, or capsule. In another embodiment, the method comprises a step of administering the said composition via topical route is in one of a dosage form of solution, cream, paste, or lotion.

In one embodiment, the method comprises a step of administering the said composition comprises one or more pharmaceutical acceptable carriers and excipient, wherein the pharmaceutically acceptable carriers and excipients is water. In one embodiment, the method comprises a step of administering of the said composition comprising sodium thiosulfate (Na₂S₂O₃) compound is effective against cancer viruses containing disulfide bonds, wherein the cancer virus containing disulfide bonds are Zika virus, papilloma virus and Epstein-Barr virus (EBV).

In one embodiment, the method comprises a step of administering the said composition comprising sodium thiosulfate (Na₂S₂O₃) compound binds with a polymerase magnesium of a viral metal and viral heavy metal, thereby disabling or killing the virus. In another embodiment, the method of administering the said composition comprising sodium thiosulfate (Na₂S₂O₃) compound releases heavy metal binding atoms and molecules. Further, the method of administering the said composition comprising sodium thiosulfate (Na₂S₂O₃) compound attacks the mucin component of the virus. In another embodiment, the method of administering the said composition comprising sodium thiosulfate (Na₂S₂O₃) provides exogenous electrons for confounding a viral disulfide bond repair. In some embodiments, the administering frequency is daily dosage in the range of 1 mg-2 g per kg body weight of the patient.

Sodium Thiosulfate, STS and Na₂S₂O₃ are used interchangeably referring to sodium thiosulfate. The term S—S bond refers to disulfide bonds, H₂S refers to Hydrogen sulphide and SO₂ refers to sulfur dioxide. The various embodiment of the present invention relates to the field of microbiology and antimicrobial pharmacotherapy. More particularly, the method of the invention relate to action of the active sulfur present in sodium thiosulfate that binds certain metals within polymerase or viral protein, or the breaking of the disulfide (S—S) bonds in glycoproteins with the resulting degradation of viral envelope structural integrity and reproductive function in the target virus.

According to an embodiment herein, the method of treatment comprises administering a composition of sulfur containing compound, which is used in solid or liquid forms. The sulfur containing compound is sodium salt of sulfur. The sulfur containing compound is sodium thiosulfate (STS). The therapeutic amount of sodium thiosulfate (Na₂S₂O₃) comprises daily dosage in the range of 1 mg-2 g per kg body weight of the human subject. The variations of sulfur have the capability of impairing the disulfide bonds in viruses. In the human body, the sodium thiosulfate releases hydrogen sulfide, sulfur dioxide and S (Sulphur atoms), and also forms SO₃-depending on the conditions. There are three sulfur atoms anticipated out of STS which eventually split S—S, digest protein amino acids, and dissolve the lethal MUCIN protein. The STS uses its H₂S primarily to bind with metals, even magnesium in Polymerase and zinc in VP-30 proteins. Furthermore, normally the S—S bonds act as hinges allowing movement in a virus, however, the virus and lipid membrane is impaired in anchoring, moving, folding or replication by S—S bond splitting. SO₂ is also a byproduct of STS under certain conditions in the body. SO₂ also can vividly break disulfide bonds in a virus or in vitro.

The sodium thiosulfate is a sulfur-based pharmaceutical agent and proven to split S—S bonds, open the blood-brain barrier of the human for cancer drugs to work and treat hypertension in pregnancy. STS is a colorless crystalline compound that is more familiar as the pentahydrate, Na₂S₂O₃.5H₂O, an efflorescent, monoclinic crystalline also called sodium hyposulfite. Sodium thiosulfate is produced chiefly from liquid waste of sodium sulfide or sulfur dye manufacture. In the laboratory, this salt is prepared by heating an aqueous solution of sodium sulfate with sulfur or by boiling aqueous sodium hydroxide and sulfuric acid according to this equation:

6NaOH+4 S→2 Na₂S+Na₂S₂O₃+3 H₂O

The human liver, kidneys and testes produce thiosulfate reductase, which catalyzes STS producing hydrogen sulfide (H2S); as follows Na₂S₂O₃ always supplies the S—S Split Atoms & Molecules: HYDROGEN SULFIDE PROOF of the use delivery of H₂S from Na₂S₂O₃ Thiosulfate is a potential respiratory electron acceptor for bacteria which live in anoxic environments or at the anoxic/oxic interface. The ability to respire thiosulfate is conferred by the enzyme thiosulfate reductase which catalyzes the reaction.

S₂O₃²⁻+2H⁺+2e⁻→HS⁻+HSO₃⁻

SO₂ is also a byproduct of Na₂S₂O₃ under certain conditions in the body. Furthermore, STS reacts with proteins, amino acids, and glycoproteins and produce hydrogen sulfide, H₂S and sulfur dioxide, SO₂, which then degrades glycoprotein, and H₂S & SO₂ continue by confusing the issue of sulfur replacement to the damaged finite disulfide bonds, by factoring infinite multiples of 6 electrons for every one sulfur damaged.

According to the embodiments herein, the safe sulfur containing compound is provided. The medically safe compound having the capability of splitting S—S bond is Na₂S₂O₃ (sodium thiosulfate, STS). At least 2 of the 3 three sulfurs coming out from STS reactions splits S—S bonds, digest protein amino acids. The self-regenerative properties of Na₂S₂O₃ to form Na₂S₄O₆, a tetrathionate is beneficial in self-regenerative available sulfur to destroy protein, bearing in mind that the human is immune to STS and is born with it as a metal eliminator and the reductase is inborn in liver, testes etc. Viruses have biochemical structures in the viral envelope, and in polymerase that are susceptible to attack by STS, or by the sulfur in H₂S and SO₂. This is as follows: the glycoprotein of which viral envelopes are composed are stabilized by disulfide (S—S) bonds. STS (SO₂ and H₂S) break these disulfide bonds, and continue disrupting the viral envelope.

In cancer virus, the polymerase must contain magnesium (Mg) in order for the virus to be infectious. Removing Mg from polymerase renders the virus non-lethal. STS by way of H₂S, which (can) capture Mg, producing MgS₂O₃ or MgS, respectively, render the polymerase inactive. The RNA tail section containing lethal kill magnesium section is called the PolyA′. In addition, zinc-binding in VP-30 is required for virus transcription to occur. STS and H₂S capture Zn, producing ZnS₂O₃ or ZnS, respectively, de-activating transcription. The disruption of zinc in Lassa fever is more profound. The study using beef broth and sulfur(s) of Na₂S₂O₃ supports active sulfur binding of added copper and gas formation. The H₂S/S/SO₂ reactions were activated in the beef broth solutions by adding copper sulfate and Na₂S₂O₃ forming CuS. The sodium thiosulfate reacts with copper sulfate to form copper monosulfide, CuS, H₂S and SO₂. The H₂S alters amino acids in rat brains. The S, H₂S, SO₂ disrupt the lipid layer and is proven to disable S—S bonds and can cause protein degradation in an independent studies.

Due to intrinsic proven production of metal-binding sulfur and production of available SO₂ and H₂S, and SO₃ permits multiple viruses to be impaired or killed for use by the medical community to target any living virus with S—S bonds or any virus using zinc as building block in the virus. Poly A is only Poly A with Magnesium.

Humans produce thiosulfate reductase in the liver, kidneys, and testes. It is also believed to be produced within the mitochondria of the human cell itself. When STS encounters this enzyme, the reduction product is hydrogen sulfide; the H₂S is the most aggressive form of binding sulfur on the earth which is human safe. STS is safe for all wildlife and it is also agriculturally safe while present in many food supplies/sources, and is unrestricted in agriculture USDA Law 2009. In humans with viral infection, the sulfur in hydrogen sulfide and in STS attacks and degrades certain biochemical components of the virus, including: dissolution of the disulfide couplets present in certain glycoproteins found in the viral envelope, thus disrupting viral envelope integrity; thus hindering viral binding with CD4 cells; binding to magnesium in polymerase disabling viral RNA replication; binding to zinc in VP-30 disabling transcription; attacking and degrading the cysteine found within the viral envelope, and lastly the attacking and disruption of S—S bond repairs in virus by STS-Electron and Sulfur interferences The presence of these multiple attack points within numerous viruses render them susceptible to disruption by STS. The glycoprotein (GP), Zinc, and S—S bonds are common denominators of most killer viruses and some cancers.

According to the embodiments herein, the safe sulfur containing compound is provided. The medically safe compound has the capability of splitting S—S bond is Na₂S₂O₃ (sodium thiosulfate, STS). At least two of the three sulfur atoms coming from STS reactions split S—S bonds, digest protein amino acids. STS binds with metals, even magnesium in Polymerase and zinc in VP-30. The lipid membrane is impaired in anchoring, moving, folding or replication by S—S bond splitting. By paving the way for mutation and badly formed SS bonds by releasing unneeded Sulfur atoms and electrons, thereby breeding confusion in the cancer virus repair mechanism process.

According to the embodiments of the present invention, sodium thiosulfate leads to the S—S disulfide bond breakage and binds with Mg polymerase and Zn of VP-30 of the virus, so that tetherin viral alarm system fails and no immune response happens. The interruption of Mg can halt RNA replication to human's lifespan advantage, the introduction of exogenous aberrant Sulfur and electrons onto the cancer virus Repair-Bench-breeds mutations.

According to an embodiment of the present invention, the sodium thiosulfate can kill viruses selected from the group consisting of herpes, dengue, zika, pox, measles, mumps rubella, or HIV, Lassa fever, Dengue fever and the cancer viruses, and solid cancer tumors a using cleavage of the diseases specialized disulfide bonds, especially. The cancer called “fibrosarcoma” and many or all solid cancer tumors other cancers treatable by H₂S/STS situations are: those having an effect in breaking ‘the disulfide bond between Cys437 and Cys542. The Cys437 and Cys542 are necessary for the secretion and activation of heparanase. The heparanase is a precursor to cancer. The overexpression of heparanase has been observed in many human tumor types, such as those in the head and neck pancreatic tumors, hepatocellular carcinoma, esophageal carcinoma and cultured human tumor cell lines

The sodium thiosulfate reacts with water and produces hydrogen disulfide (H₂S) micro portions of gas inside the human body. H₂S is referenced as being harmful to viral membrane integrity. H₂S is proven to alter the amino acids in rat brain, (autopsies available). The smaller active sulfur binds faster to the metals of viruses and breaks the S—S disulfide bonds and cause protein degradation. H₂S is referenced in 2009 Photoelectric Experiment as breaking S—S bonds. No protein, no peptide no amino acid is immune to H₂S tenacious splitting—breaking of SS bonds.

The sodium thiosulfate (Na₂S₂O₄) reacts with water (H₂O) or dilutes gastric acid of the stomach or STR sodium thiosulfate reductase to produce H₂S and SO₂. The H₂S and SO₂ molecules react with glycoproteins, degrade mucin, and hinder the viral binding with CD4 cells. The H₂S and SO₂ binds with magnesium in polymerase and disable the viral RNA replication, binds to zinc in VP-30 and disable the transcription. The cysteine found within the viral envelope is also attacked and degraded. The presence of these multiple attack points within numerous viruses renders them susceptible to disruption by STS. The S—S markings are the targets of a reducing agent and the VP-30 containing zinc, and polymerase the replicator driver containing magnesium make the cancer virus a good target for Sodium thiosulfate Na₂S₂O₃.

The bindable metal (zinc) sulfur-degradable disulfide bonds and glycoprotein-amino acids & strategically placed S—S bonds and/or lipid membranes available to H₂S & SO₂ both of which can both separate S—S bonds, and destroy amino acids protein. The disruption, and biodegradation of a viral protein matrix or a microbe or other entity is that when S—S bond breaks, it has a replacement waiting a sulfur. However, if competitive Sulfur atoms each with 8 electrons are crowding the delivery shelf, then the electron matching process is another ball game of protracted time and/or missing and non-replaced “S” parts. The confusion of choice in replacement of the broken—missing bond, will delay repair and leave a hole and vulnerability hopefully to further degradation.

The Thiol-disulfide exchange showing the linear intermediate in which the charge is shared among the three sulfur atoms. The thiolate group inhibits a sulfur atom of the disulfide bond, displacing the other sulfur atom and forming a new disulfide. A disulfide bond, also called an S—S bond, or Disulfide Bridge, is a covalent bond derived from two thiol groups. In biochemistry, the terminology R—S—S—R connectivity is commonly used to describe the overall linkages. The most common way of creating this bond is by the oxidation of sulfhydryl groups. The S of H₂S always chooses to destroy a bond, and not to bond at all to the disulfide bond. The missing sulfur can be replaced in multiple degrees such as vertical, horizontal and tangential, etc., according to the formula x/360° alignment.

Instead of an amino acid or virus replacing its losses per habit systematically, random selection is presented to the empty sulfur slot, resulting in aberrant positioning and subsequent mutation or malfunctioning amino-acids or viruses or people's muscles, or viruses which do not grow, a Herpes virus that could not grow does not hurt human very long. Further, one sulfur is never alone and can be 6+ at a time creating 48 electrons from one composite. The repair of split S—S bonds is not simple. The complexity of S replacement as referring to electron alignment and the multiplicity of S and concomitant electrons to overwhelm the mechanics of the virus play a role in damaging the virus. The use of a multiple Na₂S₂O₃ which has Sulfur tetrathionate forming sulfurs each along with 4 modes of sulfur and 6-8 electrons per sulfur poses a serious unsolvable mathematical problem to any S—S bond repair virus or microbe, etc. The humans are born with Na₂S₂O₃ and bonds we are not broken by it. if the virus or any item has a repair slot of sulfurs, any major change lower or higher will alter the mechanic so lower number are not enough to repair, too many numbers such as 10 sulfurs are hanging around from a few Na2S2O3 molecules that is 80 electrons. In all sorts of arrangements and positions to be adjusted for and the Virus or repairee cannot do it adequately. It involves the Sulfur and its 6-8 electrons positionally on Na₂S₂O₃ pose another variable in having 4 sulfurs forms available. Na₂S₂O₃ does so by adding multiple sulfur atoms i.e. 6-8 electrons/sulfur atom as variables unwanted and unexpected by the virus repair machine.

The repair in a split amino acid bond is dependent on electron alignment. Each sulfur has 8 electrons. Further, the missing slot needs just a set up to receive 8 electrons around the new sulfur. If there is misalignment of electrons in the replacing sulfur in the broken S—S bond then hesitation, confusion and delays can occur. The Na₂S₂O₃ poses another variable in having 4 sulfurs forms available. Each sulfur has 8 electrons. Further, the missing slot needs just one sulfur to align correctly, not angled not backwards, not tangential, however, figuratively-exactly flush i.e. the atom has to be horizontal. However, any selected angle would likely be the best one comprising vertical, horizontal and tangential.

The sulfur replacement of a broken-disulfide bond, S—S includes electron alignment as well. Each Sulfur has 6-8 electrons. Normally a split S—S is replaced by the S located in amino acids inventory. However, the replacement has to be just like fitting 8 holes on a wheel fitting an 8 bolt axle. If the wheel is angled or tangential or horizontal in the wrong place electrons do not match as they came off the amino acid rack ready to install. The sulfur cannot be placed in an empty hole and has 6 electrons to fit. If the variables exists as below like (A) & (B) then the empty slot for sulfur is inconvenienced and confused too many sulfurs or too many electrons. The use of a multiple Na₂S₂O₃ which has Sulfur tetrathionate forming sulfurs each along with 4 modes of sulfur and 8 electrons per sulfur poses a serious unsolvable mathematical problem to any S—S bond repair virus or microbe etc. The humans are born with STS and S—S bonds, however, the S—S bonds in humans are immune to STS.

If the virus or other item has a repair slot of sulfurs. Any major change lower or higher alters the mechanics and the low changes could not be enough to repair, too many means if 10 sulfurs are hanging around from a few Na₂S₂O₃ molecules that is 80 electrons. In all sorts of arrangements and positions to be adjusted for and the Virus or repairee cannot perform it adequately.

The size of the sodium thiosulfate atom is 0.8 nm. The size of sodium atom is 0.4 nm while the size of sulfur atom is 0.4 nm. The size of mitochondria is 1000 nm.

H₂S═H₂S═H₂S=<0.300 nm (1)

SO₂═SO₂═SO₂=<0.300 nm (2)

With respect to (1) and (2), the sizes of H₂S and SO₂ are approximately the same. The sodium thiosulfate (STS) by products combine to eventually reform STS after a series of simple steps. For example, SO₂+SO₂ will eventually reform STS after a series of simple steps. Thus, the present invention provides a method for the treatment of cancer. The present invention provides a method of administering sodium containing compound for the inhibition of the viral infection. The present invention provides a method of administering therapeutic amount of composition for the treatment, attenuation and inhibition of viruses in human body. The sodium containing compound inhibits the disulfide bonds in viruses and cancers and cause their death. The composition is easy to prepare and administer.

The monetary issue of the oral form allows affordable health and avoidance of death using the very inexpensive oral form makes the use of a vaccine possibly secondary not obsolete. No reported Fatal in 22 years of IV use for Cyanide and Pregnancy-Eclampsia. None. One oral dose or IV injection can manage many virus vs-as opposed to a vaccine one or two at best, or the MMR. For the most part, much of the expense of creating a vaccine may be spared by using an IV drug 100% safe for pregnant wife and unborn child. The Oral ingestion of STS as a food preservative or salt additive or food supplement as done in Salt Packets, seems logical and safe for sure as shown in 2009 Federal Ruling USDA “Of Total Exemption of STS” from an restriction as an agricultural additive to fertilizer.

STS safely biodegrades amino acids polypeptides proteins and glycoproteins in any virus by mean of itself, H₂S and SO₂ its main breakdown products. STS splits the bonds of any “any” S—S disulfide bond (virus infections or cancer or other) and disrupt the integrity of that item, organism, or cancer or infection. The Tetherin is the body's signal to stop intruders. Thus as per the embodiments of the present invention, the multifunction of STS is provide. The STS kills diseases in three (3) main ways: (1) applications of metal binding abilities to impair protein synthesis and RNA replication of polymerase and (2) the S—S splitting ability of Na₂S₂O₃ active ingredient H₂S & SO₂ which destroys and biodegrades all proteins. (3) The sulfur electron competitive confusion entity factor wherein Extra unneeded electrons and sulfur disrupt the REPAIR PROCESS of Viruses and Cancers.

The foregoing examples have been provided merely for the purpose of explanation and are in no way to be construed as limiting of the present concept disclosed herein. While the concept has been described with reference to various embodiments, it is understood that the words, which have been used herein, are words of description and illustration, rather than words of limitation. Further, although the concept has been described herein with reference to particular means, materials, and embodiments, the concept is not intended to be limited to the particulars disclosed herein; rather, the concept extends to all functionally equivalent structures, methods and uses, such as are within the scope of the appended claims. Those skilled in the art, having the benefit of the teachings of this specification, may affect numerous modifications thereto and changes may be made without departing from the scope and spirit of the concept in its aspects.

What is claimed is:

1. A method for treating cancer to a patient or a human in need thereof, the method comprising:
administering to the patient a pharmaceutical composition comprising a therapeutic amount of sodium thiosulfate (Na₂S₂O₃) along with at least one of a pharmaceutical carrier and a pharmaceutical excipient corresponding to the mode of administration to the patient with cancer includes gastric cancer, fibrosarcoma, pancreatic cancer, and head and neck carcinomas.

2. The method of claim 1, wherein the mode of administration is one of oral route, intravenous route, inhalation route, intra-vesical route, vaginal route, rectal route, sublingual route, ophthalmic route and topical route.

3. The method of claim 1, wherein the step of administering the said composition via intravenous route, is done by infusion of a solution comprising IV-injectable sodium thiosulfate (Na₂S₂O₃).

4. The method of claim 1, wherein the step of administering the said composition via oral route is in one of a dosage form of tablet, powder, or capsule.

5. The method of claim 1, wherein the administrating the said composition via topical route is in one of a dosage form of solution, cream, paste, or lotion.

6. The method of claim 1, wherein the administering the said composition comprises one or more pharmaceutical acceptable carriers and excipients, wherein the pharmaceutically acceptable carrier and excipient is water.

7. The method of claim 1, wherein the administering the said composition comprising sodium thiosulfate (Na₂S₂O₃) compound is effective against cancer viruses containing disulfide bonds, wherein the cancer virus containing disulfide bonds are papilloma virus and Epstein-Barr virus (EBV).

8. The method of claim 1, wherein the administering the said composition comprising sodium thiosulfate (Na₂S₂O₃) compound binds with a polymerase magnesium of a viral metal and viral heavy metal, thereby disabling or killing the virus.

9. The method of claim 1, wherein the administering the said composition comprising sodium thiosulfate (Na₂S₂O₃) compound attacks mucin component of the virus.

10. The method of claim 1, wherein the administering the said composition comprising sodium thiosulfate (Na₂S₂O₃) compound releases heavy metal binding atoms and molecules.

11. The method of claim 1, wherein the administering the said composition comprising sodium thiosulfate (Na₂S₂O₃) provides exogenous electrons for confounding a viral disulfide bond repair.

12. The method of claim 1, wherein the administering frequency is daily dosage in the range of 1 mg-2 g per kg body weight of the patient.
